(19) United States
(12) Patent Application Publication
(10) Pub. No.: US 2023/0033952 A1
(43) Pub. Date: Feb. 2, 2023

(54) CRANIOCEREBRAL OPERATION PASSAGE

(71) Applicant: CHONGQING JINYUANXI MEDICAL TECHNOLOGY CO., LTD., Chongqing (CN)

(72) Inventor: Ying LI, Chongqing (CN)

(21) Appl. No.: 17/787,846

(22) PCT Filed: Mar. 26, 2021
(86) PCT No.: PCT/CN2021/083294

§ 371 Date: Jun. 21, 2022

(30) Foreign Application Priority Data
May 12, 2020 (CN) 202010398773.5

(51) Int. Cl. A61B 17/34

(52) U.S. Cl. CPC A61B 17/3403; A61B 17/3421; A61B 2017/3405

(57) ABSTRACT

A craniocerebral operation passage is provided, including a guide device, a trocar, and an outer sheath. The guide device is used for penetrating into the brain of a patient; a connection hole is provided in the middle of the trocar and passes through the trocar in a direction of a central axis of the trocar; the connection hole and the trocar are coaxially disposed; the trocar can sheathe on a periphery of the guide device through the connection hole and can slide in a lengthwise direction of the guide device; the outer sheath is internally hollow for providing an operation passage; and the outer sheath can sheathe the periphery of the trocar. The craniocerebral operation passage is accurately located and can effectively protect brain tissues of a patient.

### CROSS REFERENCE TO RELATED APPLICATION

This patent application is a national stage application of International Patent Application No. PCT/CN2021/083294, filed on Mar. 26, 2021, which claims the benefit and priority of Chinese Patent Application No. 202010398773.5 filed on May 12, 2020, the disclosures of which are incorporated by reference herein in their entireties.

### TECHNICAL FIELD

The present disclosure relates to the technical field of medical devices, specifically to a craniocerebral operation passage.

### BACKGROUND ART

Neuroendoscopic craniocerebral operation has been an important direction for the development of minimally invasive neurosurgery. The core of this technology is to further reduce traumas of previous surgical approach, expand the exposure of a patient's condition, and increase the chance of visual resection of the lesion. Since the brain is a solid spherical tissue, it is necessary to artificially establish an operation passage in order to reach lesions in the brain parenchyma during the operation. Existing operation passages are often established through ostomy in combination with brain spatula stretching, which causes large damage to brain tissues. In addition, it is also difficult to find and locate a lesion hidden in the brain parenchyma (like digging a well in a field).

### SUMMARY OF THE INVENTION

The present disclosure aims to provide a craniocerebral operation passage to solve the above-mentioned problems of the prior art, so that the craniocerebral operation passage is accurately positioned and can effectively protect patient's brain tissues.

To achieve the above-mentioned purpose, the present disclosure provides the following solution.

The present disclosure provides a craniocerebral operation passage, comprising a guide device, a trocar, and an outer sheath. The guide device is used for penetrating into a brain of a patient; a connection hole is provided in a center of the trocar and runs through the trocar in a central axis direction of the trocar; the connection hole and the trocar are coaxially disposed; the trocar can sheathe on a periphery of the guide device through the connection hole and is able to slide in a lengthwise direction of the guide device; the outer sheath is internally hollow for providing an operation passage; and the outer sheath is able to sheathe on a periphery of the trocar.

Preferably, the trocar is a cone.

Preferably, the outer sheath is able to sheathe on the periphery of the trocar from one end at a bottom surface of the trocar.

Preferably, a side wall of the outer sheath is transparent.

Preferably, the guide device is a puncture needle.

Preferably, a tip of the puncture needle is used for puncturing the brain; the trocar sheathes on the periphery of the puncture needle through one end of the puncture needle away from the tip; and a sharp end of the trocar is disposed close to the tip of the puncture needle.

Preferably, the guide device is a neuronavigation probe.

Preferably, the trocar is further provided with a connection slot that runs through the trocar in the central axis direction of the trocar; a slot bottom of the connection slot extends to an inner wall of the connection hole; an opening of the connection slot extends to a side wall of the trocar; and the trocar can sheathe on the periphery of the neuronavigation probe through the opening of the connection slot.

Compared with the prior art, the following technical effects are achieved in the present disclosure.

In the craniocerebral operation passage provided by the present disclosure, which comprises a guide device, a trocar, and an outer sheath, the guide device is used for penetrating into a brain of the patient before an operation passage is established and reaching a lesion position. After penetrating in place, the guide device can play a guiding role in the subsequent establishment of the operation passage. The connection hole is provided in a center of the trocar and runs through the trocar in a central axis direction of the trocar, and the connection hole and the trocar are coaxially disposed. The trocar can sheathe the periphery of the guide device through the connection hole and can slide in the lengthwise direction of the guide device. When the guide device penetrates in place, the trocar slowly sheathes the periphery of the guide device and is pushed towards the brain of the patient. The sharp end of the trocar firstly contacts the brain of the patient, so that the trocar can gradually push away the brain tissues of the patient, which causes little damage to the brain tissues and makes intervention more convenient. The outer sheath is internally hollow for providing a operation passage; and the outer sheath can sheathe the periphery of the trocar. After the trocar is in place, the outer sheath slowly sheathes the trocar along the periphery of the trocar; and the guide device and the trocar withdraw, thus forming the operation passage in the outer sheath.

### BRIEF DESCRIPTION OF THE DRAWINGS

In order to describe embodiments of the present disclosure or technical solutions in the prior art more clearly, drawings required to be used in the embodiments will be briefly introduced below. It is apparent that the drawings in the descriptions below are only some embodiments of the present disclosure. Those of ordinary skill in the art also can obtain other drawings according to these drawings without making creative work.

FIG. 1 is a schematic structural diagram of a craniocerebral operation passage provided by Embodiment I;

FIG. 2 is a schematic structural diagram of a trocar in the craniocerebral operation passage provided by Embodiment I;

FIG. 3 is a schematic structural diagram of an outer sheath in the craniocerebral operation passage provided by the present application;

FIG. 4 is a schematic structural diagram of a craniocerebral operation passage provided by Embodiment II;

FIG. 5 is a schematic structural diagram of a trocar in the craniocerebral operation passage provided by Embodiment II; and

FIG. 6 is a top view of the trocar in FIG. 5.

In the drawings: 100—craniocerebral operation passage; 1—puncture needle; 2—trocar; 21—connection hole; 22—connection slot; 3—neuronavigation probe; and 4—outer sheath.

### DETAILED DESCRIPTION OF THE EMBODIMENTS

The following clearly and completely describes the technical solution in the embodiments of the present disclosure in combination with the accompanying drawings of the embodiments of the present disclosure. Apparently, the described embodiments are only part of the embodiments of the present disclosure, not all embodiments. Based on the embodiments in the present disclosure, all other embodiments obtained by those of ordinary skill in the art without creative work shall fall within the protection scope of the present disclosure.

The present disclosure aims to provide a craniocerebral operation passage, so as to solve the technical problems that the existing craniocerebral operation passage cannot be accurately located and causes a large craniocerebral injury.

In order to make the above-mentioned purposes, features and advantages of the present disclosure more obvious and understandable, the present disclosure is further described in detail below with reference to the accompanying drawings and specific embodiments.

### Embodiment I

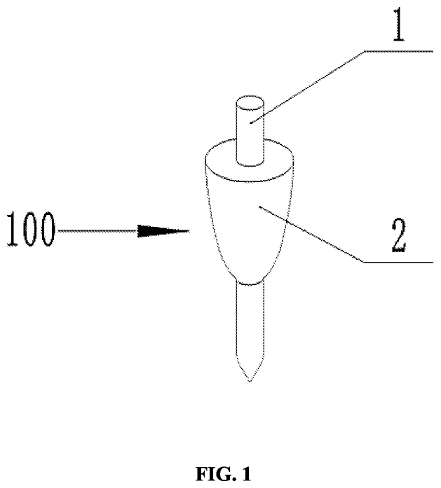

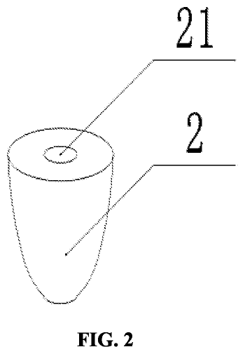

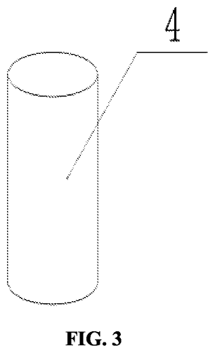

As shown in FIG. 1-FIG. 3, the present embodiment provides a craniocerebral operation passage 100, including a guide device, a trocar 2, and an outer sheath 4. The guide device is used for penetrating into the brain of a patient before an operation passage is established and reaching a lesion position. After penetrating in place, the guide device can play a guiding role in the subsequent establishment of the operation passage. A connection hole 21 is provided in the center of the trocar 2 and runs through the trocar 2 in a central axis direction of the trocar 2, and the connection hole 21 and the trocar 2 are coaxially disposed. The trocar 2 can sheathe the periphery of the guide device through the connection hole 21 and can slide in the lengthwise direction of the guide device. When the guide device penetrates in place, the trocar 2 slowly sheathes on the periphery of the guide device and is pushed towards the brain of the patient. The sharp end of the trocar 2 firstly contacts the brain of the patient, so that the trocar 2 can gradually push away the brain tissues of the patient, which causes little injury to the brain tissues and makes intervention more convenient. The outer sheath 4 is internally hollow for providing an operation passage; and the outer sheath 4 can sheathe the periphery of the trocar 2. After the trocar 2 is in place, the outer sheath 4 slowly sheathes on the trocar 2 along the periphery of the trocar 2. Then the guide device and the trocar 2 withdraw, thus forming the operation passage in the outer sheath 4.

Specifically, the trocar 2 is a cone, which ensures that an outer circumferential surface of the trocar 2 is smoothly transitioned. When the trocar 2 gradually pushes away the brain tissues, the brain tissues of the patient can be effectively protected. More preferably, a bottom surface of the trocar 2 is also smoothly transitioned to the trocar tip. In an actual use process, if a passage with a large inner diameter is desired, trocars 2 of multiple specifications can be used cooperatively. That is, a small-size trocar 2 slowly penetrates into the brain tissues at first, then withdraws, and is sequentially replaced with a sequence of trocars 2 with increasing sizes, so as to be able to gradually push away the brain tissues to a desired range to meet operation requirements and avoid a certain injury to the brain tissues, caused by sudden distraction of the brain tissues of the patient. Furthermore, in the actual production process, a withdraw device may be arranged on a bottom surface of the trocar 2, such as a sheet-like body or a ring-like body, as long as it is convenient for assisting in the withdrawal of the trocar 2 through tweezers or other devices.

The outer sheath 4 can sheathe on the periphery of the trocar 2 from one end of the bottom surface of the trocar 2. Thus, after the trocar 2 pushes away the brain tissues, the outer sheath 4 enters the brain tissues along the periphery of the trocar 2 to provide a passage for craniocerebral operation.

A side wall of the outer sheath 4 is transparent, which is convenient for observing a push-in position and realizing a clear field of view.

The guide device is a puncture needle 1. The puncture needle 1 is an ordinary operation puncture needle 1, one end of which is a sharp end. In the process of penetrating into the brain, the resistance is relatively low, and penetration is facilitated.

The tip of the puncture needle 1 is used for puncturing the brain. The trocar 2 sheathes on the periphery of the puncture needle 1 through one end of the puncture needle 1 away from the tip. Furthermore, the sharp end of the trocar 2 is close to the tip of the puncture needle 1. By means of the guiding effect of the puncture needle 1, the trocar 2 is pushed into the brain along the puncture needle 1. The sharp end of the trocar 2 contacts the brain of the patient at first and is gradually pushed into the brain of the patient, so that the trocar 2 gradually pushes away the brain tissues, which reduces the resistance and protects the brain tissues of the patient.

### Embodiment II

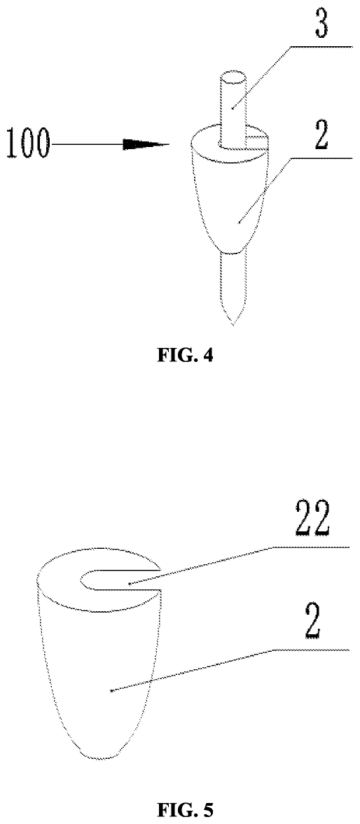

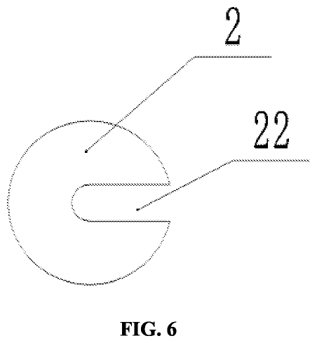

As shown in FIG. 4 to FIG. 6, a difference between the present embodiment and Embodiment I is: the guide device is a neuronavigation probe 3. Neuronavigation is an accurate locating technology for designing an operation solution before an surgical operation and guiding operation in real time during the surgical operation. Accurate location can be realized through the neuronavigation probe 3.

The trocar 2 is further provided with a connection slot 22 and runs through the trocar 2 in a central axis direction of the trocar 2, and the slot bottom of the connection slot 22 extends to an inner wall of the connection hole 21. An opening of the connection slot 22 extends to a side wall of the trocar 2, and the trocar can sheathe on the periphery of the neuronavigation probe 3 through the opening of the connection slot 22. Since an end of the neuronavigation probe 3 away from the tip is provided with other equipment, pushing-in of the trocar 2 cannot be realized by directly passing the neuronavigation probe 3 through the connection hole 21. When the trocar 2 and the neuronavigation probe 3 are used cooperatively, the trocar 2 may sheathe on the periphery of the neuronavigation probe 3 from the side wall of the neuronavigation probe 3 through the connection slot 22 on the trocar 2 and is then gradually pushed towards the brain. After puncturing in place, the neuronavigation probe 3 is withdrawn; the outer sheath 4 sheathes on the periphery of the trocar 2; and the trocar 2 is then withdrawn, thus establishing the craniocerebral operation passage 100.

The principle and embodiments of the present invention are described by applying specific examples in the present specification. The descriptions of the above embodiments are only intended to help to understand the method of the present invention and a core idea of the method. In addition, those ordinarily skilled in the art can make changes to the specific implementation modes and the application scope according to the idea of the present invention. From the above, the contents of the specification shall not be deemed as limitations to the present invention.

1.-8. (canceled)

9. A craniocerebral operation passage, comprising:
a guide device, wherein the guide device is used for penetrating into a brain of a patient;
a trocar; and
an outer sheath,
a connection hole is provided in a center of the trocar and runs through the trocar in a central axis direction of the trocar;
wherein the connection hole and the trocar are coaxially disposed;
wherein the trocar is configured to sheathe on a periphery of the guide device through the connection hole and is able to slide in a lengthwise direction of the guide device;
wherein the outer sheath is internally hollow for providing an operation passage; and the outer sheath is able to sheathe on a periphery of the trocar.

10. The craniocerebral operation passage according to claim 9, wherein the trocar is a cone.

11. The craniocerebral operation passage according to claim 9, wherein the outer sheath is able to sheathe on the periphery of the trocar from one end at a bottom surface of the trocar.

12. The craniocerebral operation passage according to claim 9, wherein a side wall of the outer sheath is transparent.

13. The craniocerebral operation passage according to claim 9, wherein the guide device is a puncture needle.

14. The craniocerebral operation passage according to claim 13, wherein a tip of the puncture needle is used for puncturing the brain; the trocar sheathes on the periphery of the puncture needle through one end of the puncture needle away from the tip; and a sharp end of the trocar is disposed close to the tip of the puncture needle.

15. The craniocerebral operation passage according to claim 9, wherein the guide device is a neuronavigation probe.

16. The craniocerebral operation passage according to claim 15, wherein the trocar is further provided with a connection slot that runs through the trocar in the central axis direction of the trocar; a slot bottom of the connection slot extends to an inner wall of the connection hole; an opening of the connection slot extends to a side wall of the trocar; and the trocar is able to sheathe on the periphery of the neuronavigation probe through the opening of the connection slot.
